(19) United States
(12) Patent Application Publication
(10) Pub. No.: US 2023/0111182 A1
(43) Pub. Date: Apr. 13, 2023

(54) METHOD FOR A PREDICTIVE PROGNOSIS OF MENOPAUSE ONSET

(71) Applicant: ALLELICA S.R.L., Roma (IT)

(72) Inventors: Paolo DI DOMENICO, Roma (IT); Alessandro BOLLI, Roma (IT); Giordano BOTTA', Roma (IT)

(21) Appl. No.: 17/802,724

(22) PCT Filed: Jan. 28, 2021
(86) PCT No.: PCT/IB2021/050668

§ 371 Date: Aug. 26, 2022

(30) Foreign Application Priority Data
Feb. 26, 2020 (IT) 102020000003988

(51) Int. Cl. C12Q 1/6883; A61B 5/00; G16H 50/30

(52) U.S. Cl. CPC C12Q 1/6883; A61B 5/4306; A61B 5/7267; A61B 5/7275; G16H 50/30; C12Q 2600/156

(57) ABSTRACT

A method is for predictive prognosis of a woman's menopause onset. The method includes accessing the Single Nucleotide Polymorphisms (SNPs) of the woman; processing the woman's genetic data, to provide a predictive prognosis of menopause onset in relation to the phenotype. The phenotype includes an age group/limit with respect to the predictive prognosis, or indication of a woman's likely age for menopause onset. The processing includes identifying a predetermined set and subset of SNPs associated with the phenotype. Each of the SNPs the set includes an identifier of SNPs, and is associated with a respective pre-calculated first relevance parameter. A first value of polygenic risk score is calculated based on the first personalized subset of SNPs and respective first pre-calculated relevance parameters. The predictive prognosis of menopause onset relative to each phenotype is determined based on the polygenic risk score.

### TECHNOLOGICAL BACKGROUND OF THE INVENTION

### Field of Application

The present invention relates to a method for a predictive prognosis of a woman's menopause onset.

The general technical field of the present invention is therefore that of predictive methods, performed by means of electronic computation, used in the medical field to support predictive prognoses.

### Description of the Prior Art

The average age of women upon their first birth in Western countries is constantly increasing. This has given rise to numerous problems in the field of fertility and reproductive life planning, particularly for women.

In fact, female fertility already significantly decreases ten years before menopause; therefore, being able to predict the age of menopause becomes a fundamental factor in planning reproductive life.

Currently, the amount of AMH (antimüllerian hormone) is used as a predictor. However, such a predictor is successful to provide predictive value only in the time range of a few years, thus proving to be of little use in supporting the planning of a woman's reproductive life.

Furthermore, the link between genetics and menopause is widely documented in the literature, particularly in connection with the phenomenon of early menopause (before the age of 45) and ovarian failure (POI).

A study known as GWAS (Genome Wide Association Study) used 2,418,696 Single Nucleotide Polymorphisms (SNPs) to investigate the genetic components involved in determining the age of naturally occurring menopause (NMA). This study produced a “summary statistics” (SS) indicating the Single Nucleotide Polymorphisms (SNPs) and providing them with a score of significance (“P-Value” parameter) and an effect (13 parameter) as a function of the association detected.

This type of study only evaluates the statistical association between SNP and the age of menopause onset, without analyzing the predictive capacity of the result obtained; this limitation makes the GWAS not directly applicable in clinical practice.

Also for this reason, there is currently no reliable measure which can directly associate the genetic risk (relative risk) of early menopause or is capable of associating such risk with other existing risk factors (absolute risk).

Therefore, there is still a strong need to devise prognostic methods capable of providing more reliable, accurate and precise results, especially with regard to the correlation of the identified risk with different phenotypes, for example the expected age or age group of menopause onset or with other phenotypes.

### SUMMARY OF THE INVENTION

It is the object of the present invention to provide a method for a predictive prognosis of the onset of a woman's menopause, which allows to obviate at least partially the drawbacks mentioned above with reference to the known art, and to respond to the aforementioned needs particularly felt in the technical field considered. Such an object is achieved by a method according to claim 1.

Further embodiments of such a method are defined by claims 2-22.

### BRIEF DESCRIPTION OF THE DRAWINGS

Further features and advantages of the method according to the invention will become apparent from the following description of preferred embodiments, given by way of non-limiting example, with reference to the accompanying drawings, in which:

FIGS. 1-3 are “fraction of cases” diagrams showing the fraction of individuals who have the phenotype described in relation to calculated percentiles of PRS; FIGS. 1, 2, 3 refer to the phenotypes “menopause before age 40”, “menopause within age 45” and “menopause after age 55”, respectively;

FIGS. 4, 5 and 6 are ROC-AUC diagrams (referring respectively to the phenotypes “menopause before age 40”, “menopause within age 45”, “menopause after age 55”) obtained in a validation process of the method according to the invention;

FIGS. 7-9 are population distribution diagrams showing the number of people who have experienced menopause onset at a certain known age, partitioned by percentile of PRS, calculated according to the present method; FIGS. 7, 8, 9 refer respectively to PRS calculated for the phenotypes of menopause before age 40, menopause within age 45 and menopause after age 55;

FIG. 10 shows a cumulative probability curve of menopause onset for a woman, estimated according to the present method, compared with a cumulative probability curve of menopause onset referred to the average population;

FIG. 11 shows an example of the dependence of the absolute risk of Early Menopause (before age 45) on the percentile of PRS, as calculated by the present method, reporting two risk curves referring to two types of women (smokers and non-smokers with a Normal Body Mass Index BMI, above 18.6), in which the absolute risk was computed through a Cox proportional hazards model.

### DETAILED DESCRIPTION

A computer-implemented method for a predictive prognosis of the onset of menopause in a woman is described.

The method comprises the steps of accessing woman's genetic data comprising Single Nucleotide Polymorphisms SNPs of the woman; then, processing the aforesaid woman's genetic data, to obtain a predictive prognosis of menopause onset in relation to at least one phenotype F_{Dj}, F_{Ck}; finally, providing as a result the aforesaid predictive prognosis of menopause onset in relation to the at least one phenotype.

The aforesaid at least one phenotype F_{Dj}, F_{Ck} comprises at least one age group or an age limit with respect to which the predictive prognosis of menopause onset is to be carried out, or an indication of a woman's age at which the menopause onset is more likely.

The aforesaid step of processing woman's genetic data comprises, for each of the phenotypes considered F_{Dj}, F_{Ck}, the following steps:
identifying, in the woman's genetic data, a first personalized subset SI₁ of Single Nucleotide Polymorphisms SNPs which also belong to a first predetermined set I₁, associated with the phenotype considered, each of the Single Nucleotide Polymorphisms SNPs of such a first predetermined set I₁ comprises an identifier of Single Nucleotide Polymorphisms SNPs, and is associated with a respective pre-calculated first relevance parameter β_{1(Fn),i};
calculating a first value PRS1(Fₙ) of polygenic risk score PRS, based on the first personalized subset SI₁ of Single Nucleotide Polymorphisms SNPs and the respective first pre-calculated relevance parameters β_{1(Fn),i};
determining the predictive prognosis of menopause onset in relation to each of the phenotypes considered F_{Dj}, F_{Ck}, based on the respective first value PRS1(Fₙ) of polygenic risk score.

The method provides that the determination of the first predetermined set I₁ of Single Nucleotide Polymorphisms SNPs and the calculation of the first relevance parameters β_{1(Fn),i} are carried out in a preliminary training step, which is independent from the aforesaid step of accessing woman's genetic data and prior to the aforesaid step of processing the woman's genetic data.

Such a preliminary training step comprises training at least one algorithm (A) by means of machine learning and/or artificial intelligence techniques, based on known data.

According to an embodiment of the method, the aforesaid training is carried out based on a known dataset containing genetic data of Single Nucleotide Polymorphisms (SNPs) of women whose menopause onset age is known.

According to an embodiment of the method, the aforesaid identifier of Single Nucleotide Polymorphisms SNPs comprises a genetic variant address and an effective allele AL₁ present in such a genetic variant address; furthermore, the aforesaid first custom subset SI₁ comprises Single Nucleotide Polymorphisms SNPs in which the effective allele is traced as present in the respective genetic variant address, in the woman's genetic data, and is associated with a respective allele dosage.

In such a case, the aforesaid step of calculating a first value PRS1(Fₙ) of polygenic risk score PRS comprises summing up the value of all the pre-calculated first relevance parameters β_{1(Fn),i} associated with all the respective Single Nucleotide Polymorphisms SNPs of the first personalized subset SI₁, each multiplied by the respective allele dosage of the effective allele.

In other words, the processing of the biological data acquired from the woman allows to obtain a summary score, i.e., the aforesaid PRS, of the genetic risk composed of the sum of the risk value associated with each SNP.

According to an implementation option, such a PRS score is computed by multiplying the allelic dosage of the risk allele by a weight that is pre-calculated based on trained algorithms, which will be exemplified in more detail in the following of this description.

With reference to the multiplication by the “allelic dosage”, such an operation involves multiplying the relevance parameter, associated with a certain SNP, by the number of effective alleles present in the respective genetic variant address.

According to the most common implementation options, the “allele dosage”, i.e., the number by which the relevance parameter is multiplied, is 0 or 1 or 2.

It should be noted that the term “effective allele” (used most often by technical experts in the field) can also be referred to as “risk allele”.

In accordance with an embodiment of the method, the aforesaid at least one phenotype comprises a plurality of phenotypes Fₙ.

Such a plurality of phenotypes comprises at least one binary phenotype F_{Dj} and at least one continuous phenotype F_{Ck}.

The at least one binary phenotype F_{Dj} comprises at least one age group or an age limit with respect to which the prognosis of menopause onset or not is to be determined (or the probability is to be calculated), and the at least one continuous phenotype F_{Ck} comprises an indication of a woman's age at which the menopause onset is expected as more likely.

In such a case, the step of determining the predictive prognosis of menopause onset comprises determining the predictive prognosis of menopause onset in relation to each of the aforesaid at least one binary phenotype F_{Dj} and at least one continuous phenotype F_{Ck}, based on the respective first value PRS1(Fₙ) of polygenic risk score.

Thus, the step of providing as a result the predictive prognosis of menopause onset comprises providing the predictive prognosis of menopause onset both in relation to each of the binary phenotypes considered P(F_{Dj}), and in relation to the continuous phenotype considered or to each of the continuous phenotypes considered P(F_{Ck}).

According to an embodiment of the method, the results of the predictive prognosis comprise a menopause onset probability P(F_{Dj}), P(F_{Ck}) in relation to at least one phenotype (F_{Dj}, F_{Ck}).

In such a case, the step of determining a predictive prognosis comprises calculating a menopause onset probability P(F_{Dj}), P(F_{Ck}) with respect to each of the phenotypes considered (F_{Dj}, F_{Ck}).

The step of providing as a result the predictive prognosis of menopause onset comprises providing as a result of the prognosis the calculated menopause onset probability P(F_{Dj}), P(F_{Ck}), in relation to the at least one phenotype.

In other words, this embodiment of the method allows to obtain a menopause onset probability in an age group or age limit (first option) or an indication of an age of the woman in which menopause onset will be more likely (second option). The menopause onset age is therefore defined along with the phenotype under study. The phenotype can be binary, in the first option, indicated by F_{Dj}, based on the age chosen as the threshold, or continuous in the second option, indicated by F_{Ck}.

The binary phenotype can take the values 0 or 1, indicating, as one of the prognosis results, whether or not menopause occurs within the age threshold associated with the phenotype, or within the age group between two thresholds associated with the phenotype.

The continuous phenotype instead indicates a value corresponding to the most likely menopause onset age, according to the predictive prognosis of the method. In this regard, from a terminological point of view, the phenotype which includes age is, strictly speaking, discrete, because age is expressed in years which have at most a finite number of decimal digits; however, the term “continuous phenotype” (for easier distinction from the “binary phenotype” above) is often used, and clearly understood in the aforesaid terms, by those skilled in the art.

According to different implementation options of the method, the at least one binary phenotype comprises one or more phenotypes belonging to the following group:
menopause onset before age 40, also defined, in the literature and by those skilled in the art, “Primary Ovarian Insufficiency” (POI);
menopause onset before age 45, also defined, in the literature and by those skilled in the art, “Early Menopause”;
menopause onset after age 55, also defined, in the literature and by those skilled in the art, “Late Menopause”.

As is apparent from the above description, the method can be applied in principle to phenotypes associated with any age group, defined by any reasonable age threshold or age range.

The method implementation option described above refers to the age groups for which the prediction is usually considered more significant (different from the 45-55 age group of average menopause onset).

According to other implementation options, the method employs phenotypes corresponding to the age group 45 to 55, or any other age group for which a prediction is desired.

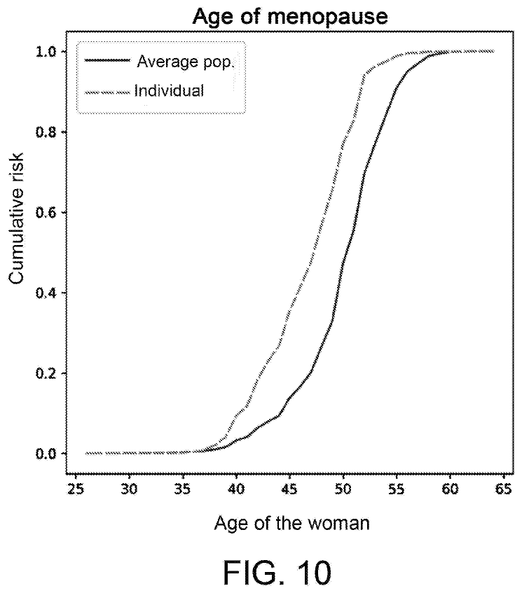

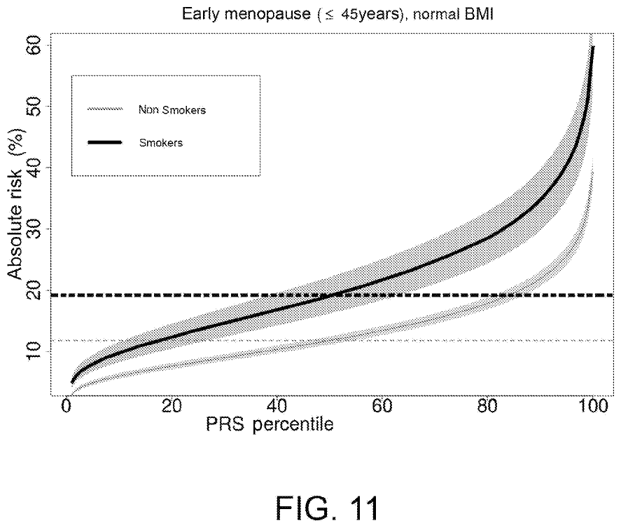

According to other implementation options of the method, the at least one continuous phenotype comprises an indication of the age at which the menopause onset is estimated as more likely and/or a distribution of menopause onset probability in relation to each year of the woman's age within a predetermined age range, and/or a cumulative probability distribution of menopause onset as a function of age (as shown for example in FIG. 10).

According to other implementation options of the method, the results of the menopause onset prognosis comprise other predictive information of menopause onset, which will be exemplified in a subsequent part of this description.

In accordance with an embodiment, the method comprises, after the step of identifying a first personalized subset SI₁ of Single Nucleotide Polymorphisms SNPs, a step of selecting a first group G₁ of Single Nucleotide Polymorphisms SNPs, belonging to the woman's first personalized subset SI₁. Such a first group G₁ comprises a first number N₁ of Single Nucleotide Polymorphisms SNPs recognized as the most relevant ones, based on a predetermined criterion.

In such a case, the step of calculating a first value PRS1 (Fₙ) of polygenic risk score PRS comprises calculating the first value PRS1(Fₙ) of polygenic risk score PRS based on the aforesaid first group G₁ of Single Nucleotide Polymorphisms SNPs and respective pre-calculated first relevance parameters β_{1(Fn),i}.

With reference to the aforesaid criterion for identifying the most relevant SNPs, the method includes various implementation variations, for example:

(i) identifying as the most relevant SNPs those SNPs which are associated with the highest relevance parameter values β_{1(Fn),i}; or

(ii) testing different PRS calculated on different SNPs, validating the PRS on known populations and choosing those SNPs which result in a better predictivity using as a metric the “sensitivity”, i.e., the ability to identify people affected by the disease as people at risk, or the “specificity”, i.e., the ability to identify unaffected people as people not at risk; or

(iii) applying methodologies of the AUC-ROC type (Area Under the Receiver Operator Characteristic Curve—ability to distinguish cases from controls) per se known; further details on these aspects will be illustrated below, regarding the validation methods of the developed models.

In accordance with an embodiment, the method provides operating with two PRS scores, thus obtaining two polygenic risk score values.

In such a case, the method comprises the further step of identifying, in the woman's genetic data, a second personalized subset SI₂ of Single Nucleotide Polymorphisms SNPs which also belong to a second predetermined set I₂, associated with the phenotype considered; each of the Single Nucleotide Polymorphisms SNPs of such a second predetermined set I₂ comprises an identifier of Single Nucleotide Polymorphisms SNPs, and is associated with a respective pre-calculated second relevance parameter β_{1(Fn),i}; the identifier of Single Nucleotide Polymorphisms SNPs comprises, also in this case, a genetic variant address and an effective allele AL₂ present in such a genetic variant address.

The method then involves calculating a second value PRS2(Fₙ) of polygenic risk score PRS, based on the aforesaid second personalized subset SI₂ of Single Nucleotide Polymorphisms SNPs and the respective pre-calculated second relevance parameters β_{1(Fn),i}.

In such a case, the step of calculating the menopause onset probability comprises calculating the menopause onset probability in relation to each of the phenotypes considered F_{Dj}, F_{Ck}, based on the respective first value PRS1(Fₙ) of polygenic risk score and/or the respective second value PRS2(Fₙ) of polygenic risk score. In particular, the menopause onset probability in relation to at least one phenotype of said at least one phenotype (F_{Dj}, F_{Ck}) is calculated based on both the respective first value (PRS1 (Fₙ)) of polygenic risk score and the respective second value (PRS2(Fₙ)) of polygenic risk score.

The determination of the second predetermined set I₂ of Single Nucleotide Polymorphisms SNPs and the calculation of the second relevance parameters β_{2(Fn),i} are carried out in a preliminary training step, which is independent from the aforesaid step of accessing woman's genetic data and prior to the aforesaid step of processing the woman's genetic data. Such a preliminary training step comprises training at least one algorithm (A) by means of machine learning and/or artificial intelligence techniques, based on known data.

According to an implementation option, given a certain phenotype, one or the other of the PRS values is used, thus the result depends on the first PRS value or the second PRS value. The choice is made during the preliminary training and/or model validation step.

According to an implementation option, logistic regression techniques or a Cox proportional hazard model, known per se, are used to build the model used in the present method, in which the value of the PRS chosen is used as a covariate.

According to another implementation option, given a certain phenotype, both PRS values are used, i.e., a combination of the two PRS, and therefore the result depends on both the first PRS value and the second PRS value. The combination is carried out in the preliminary training and/or model validation step.

In such a case, the above logistic regression techniques or a Cox proportional hazards model can be used, in which both specific PRS values of the individual are used as covariates.

In accordance with a possible implementation variant, the method comprises, after the step of identifying a second personalized subset SI₂ of Single Nucleotide Polymorphisms SNPs, a step of selecting a second group G₂ of Single Nucleotide Polymorphisms SNPs, belonging to the woman's second custom subset SI₂. Such a second group G₂ of SNP comprises a second number N₂ of Single Nucleotide Polymorphisms SNPs recognized as the most relevant ones based on a predetermined criterion (examples of such a criterion are the same as previously shown with reference to the first group G₁ of Single Nucleotide Polymorphisms SNP).

In such a case, the step of calculating a second value PRS2(Fₙ) of polygenic risk score PRS comprises calculating the second value PRS2(Fₙ) of polygenic risk score PRS based on the second group G₂ of Single Nucleotide Polymorphisms SNPs and the respective second pre-calculated relevance parameters β_{1(Fn),i}.

According to an implementation example, the aforesaid first number N₁ of relevant SNPs is at least 10; and the aforesaid second number N₂ of relevant SNPs is at least 10.

According to another implementation example, the aforesaid first number N₁ of relevant SNPs is at least 20; and the aforesaid second number N₂ of relevant SNPs is at least 20.

In accordance with an embodiment of the method, the polygenic risk scores PRS1(Fₓ), PRS2(Fₓ) calculated for one of the discrete phenotypes Fₓ are used as additional information to establish the menopause onset probability referred to another phenotype F_{y} together with the polygenic risk scores calculated for such a phenotype F_{y}, according to any combination of phenotypes Fₙ and polygenic risk scores (PRS1(Fₘ), PRS2 (Fₘ).

In such an embodiment, the “crossed” use of PRS parameters, between different phenotypes, can allow a refinement and an improvement of the predictive results, with respect to the results obtainable based on the PRS related to the single phenotype alone.

In such a case, in addition to using several PRS as covariates, the PRS are also used in combination. For example, to calculate the risk of menopause onset before age 45, the percentile in which the woman is located is analyzed in the PRS associated with the phenotype “before age 45 (<45)” and in the phenotype “after age 55 (>55)”.

For example, for a 35-year-old woman, “PRS <45=99 percentile” and “PRS>55=1 percentile” is obtained, the menopause risk before age 45 is evaluated as 35% (which represents the fraction of cases of menopause onset before age 45 which is in the last percentile of PRS<45 and in the first percentile of PRS>55). Such a result derives from the fact of analyzing two “opposite” phenotypes: therefore, if a woman has a very high risk for one phenotype and very low for the “opposite” one, the absolute risk increases.

According to an embodiment of the method, the preliminary training step comprises building the first predetermined set I₁ of Single Nucleotide Polymorphisms SNPs by means of a selection of relevant Single Nucleotide Polymorphisms SNPs carried out through the following steps: identifying Single Nucleotide Polymorphisms SNPs statistically associated with the phenotype through a genetic association study, in which each of such identified Single Nucleotide Polymorphisms SNPs is associated with a respective initial known relevance parameter β; then, identifying optimal values of first relevance parameters β_{1(Fn),i} adapted to optimize the predictive efficacy of the first polygenic risk score PRS value.

The optimization of the predictive efficacy of the first polygenic risk score PRS value is carried out from the aforesaid identified Single Nucleotide Polymorphisms SNPs and respective known initial relevance parameters (β), through the single or combined use of one or more predictive algorithms (A).

Each of such predictive algorithms is trained based on a set of known data (hereinafter also referred to as “dataset”, according to a common term in the field) containing genetic data of Single Nucleotide Polymorphisms SNPs of women whose menopause onset age is known.

The training then is concluded with the steps of defining the aforesaid first predetermined set I₁ of Single Nucleotide Polymorphisms SNPs based on the identified Single Nucleotide Polymorphisms SNPs; and defining as the respective first relevance parameters β_{1(Fn),i} the aforesaid respective optimal values of first relevance parameters β_{1(Fn),i} that are identified.

According to various possible implementation options, the aforesaid one or more trained algorithms (A) comprise one or more of the following predictive algorithms:
Clumping+Thresholding;
LD-Pred;
Stacked Clumping+Thresholding (SCT).

With reference to the aforesaid genetic association study, an implementation option of the method involves using the aforesaid study known as GWAS (Genome Wide Association Study).

More specifically, in application examples of the method actually implemented, a known set of 2,418,696 Single Nucleotide Polymorphisms (SNPs) identified by GWAS was used.

Such a GWAS study regarding menopause age used the aforesaid 2,418,696 Single Nucleotide Polymorphisms (SNPs) to investigate the genetic components involved in determining the age of naturally occurring menopause (NMA). This study produced a summary statistics SS indicating the SNPs and providing them with a score of significance (P-Value) and an effect (β) as a function of the association detected.

The aforesaid GWAS study evaluates the statistical association between SNP and phenotype without, however, taking into account the final predictivity of the parameters and P-value thus calculated. Such a study can therefore be seen as a possible starting point on which the previously illustrated developments of the method of the present invention are grafted.

It is apparent to those skilled in the art that the method disclosed herein can also be carried out starting from genetic association studies different from the aforementioned GWAS study.

Based on the genetic association study, the present method calculates the Polygenic Risk Score (PRS) representative of a score calculated as a weighted sum of the dosage of the alleles present in an individual. The weights for each allele were determined by the algorithm or algorithms used in the method, by means of a processing which starts from the weights defined in the GWAS summary statistics and leads to the calculation of new weights, characteristic of the present method.

In fact, among the intermediate results of the method, in addition to the main result consisting of the custom prognoses outcomes, there are tables representing the aforesaid first and/or second predetermined set I₁, and/or I₂, of SNPs, in which, for each SNP identified as relevant, the genetic variant address, the effective allele and the respective relevance parameter, or weight β_{1(Fn),i} are provided, conveniently calculated according to the present method.

However, it should be noted that the scope of the method goes far beyond the individual tables, both because it is applicable to various phenotypes (thus leading to different possible tables, each associated with a desired phenotype) and because it is applicable to different populations (thus leading, given a phenotype, to different possible tables, each associated with a different population considered).

With reference to the latter aspect, it is known that a score such as the PRS has a higher predictive value if calculated on an individual with the same ancestral derivation as the individuals based on which the PRS was derived, i.e., the individuals of the database with which the method was trained.

A difference between populations of different ancestrality lies, for example, in the difference in the Linkage Disequilibrium maps, which affects the determination of the causal SNPs.

With regard to the known dataset containing genetic data of Single Nucleotide Polymorphisms SNPs of women whose menopause onset age is known, one example of the method, actually implemented, uses the “UK BioBank” database, which is the largest global genetic database, within the project: “Validating genomic trait prediction algorithms for commercial use with the UK BioBank resource”.

The “UK BioBank” database (https://www.ukbiobank.ac.uk/) contains the genetic information of over 500,000 volunteers, providing clinical information. Hence, in the example shown here, the PRS has a higher predictive validity for Caucasians.

However, it is again underlined that the method described here is agnostic with respect to the population analyzed and is applicable to data related to any type of population/ancestrality, as long as the algorithms used are trained based on a known database related to such a population/ancestrality.

It should also be noted that, by applying the method starting from a population of different ethnicity, specific predictors would be obtained for the population on which the PRS was derived, i.e., different tables and different weight parameters.

With regard to the algorithms mentioned above (algorithms or predictive algorithms, adapted to be trained), that are used in a non-trivial manner in the present method, the following information is provided.

LD-Pred (Linkage Disequilibrium—Predictor) is a known algorithm, illustrated in the literature, for example in the article: B. J. Vilhjalmsson et al. “Modeling Linkage Disequilibrium Increases Accuracy of Polygenic Risk Scores”—The American Journal of Human Genetics 97, 576-592, Oct. 1, 2015.

SCT (Stacked Clumping+Thresholding) is a per se known algorithm, illustrated in the literature, for example in the article: Florian Prive et al. “Making the most of Clumping and Thresholding for polygenic scores”—The American Journal of Human Genetics—DOI: https://doi.org/10.1016/j.ajhq.2019.11.001.

Clumping+Thresholding is a per se known algorithm, illustrated in the literature, for example in the aforementioned article by Florian Prive and others and in the bibliography cited in such an article.

According to an embodiment of the method, the aforesaid at least one predictive algorithm A comprises a first trained LD-Pred algorithm (A1), configured to perform, based on a preventive training procedure, and for each of the phenotypes Fₙ, the following steps:
determining the first predetermined set I₁ of Single Nucleotide Polymorphisms SNPs, containing the first plurality of Single Nucleotide Polymorphisms SNPs identified as relevant with respect to the menopause onset, based on a known set of Single Nucleotide Polymorphisms SNPs identified by GWAS (Genome Wide Association Study);
calculating the first relevance parameters β_{1(Fn),i}, taking into account a degree of association (Linkage Disequilibrium) between the Single Nucleotide Polymorphisms SNPs and a parameter (p) representative of the fraction of Single Nucleotide Polymorphisms (SNPs) identified as causal for the phenotype considered; for each of the first parameters β_{1(Fn),i}, a plurality of values β_{1(Fn),i}(ρ_{w}) are calculated, each of which corresponds to a respective parameter value representative of the SNP fraction (ρ_{w}), representative of a fraction of random variants used, belonging to a plurality of predetermined values of such a parameter ρ_{w};
calculating the first value PRS1(Fₙ, ρ_{w}) of polygenic risk score for each of the values ρ_{w} of the parameter representative of the fraction of SNP (ρ), as a weighted sum of the alleles (AL′ᵢ) corresponding to the Single Nucleotide Polymorphisms (SNPs) of the first custom subset SI₁, each weighted by the respective first parameter β_{1(Fn),i}(ρ_{w}).

According to an implementation option, the method comprises the further steps of providing the processing results by means of a plurality of first tables T_{w}(ρ_{w}) containing, for each of the Single Nucleotide Polymorphisms SNPsᵢ, the respective identifier ID_SNPᵢ, the respective effective allele or the respective two effective alleles (AL′_{1,j}, AL′_{2,i}) and the respective first calculated relevance parameter β_{1(Fn),i}(ρ_{w}), in which each of the first tables T_{w}(ρ_{w}) is associated with a different value (pw) of the correlation parameter (ρ); and then evaluating the predictive effectiveness, or efficacy, of each of the first values PRS1(Fₙ, ρ_{w}) of calculated polygenic risk score.

The predictive effectiveness, or efficacy, is estimated, as illustrated above, based on training and comparison with data belonging to known “datasets”.

According to another embodiment of the method, the aforesaid at least one predictive algorithm A comprises at least a second trained SCT algorithm (A2), configured to perform, based on a preventive training procedure, and for each of the phenotypes Fₙ, the following steps:
determining the second predetermined set I₂ of Single Nucleotide Polymorphisms SNPs, containing the first plurality of Single Nucleotide Polymorphisms SNPs identified as relevant with respect to the menopause onset, based on a known set of Single Nucleotide Polymorphisms (SNPs) identified by GWAS (Genome Wide Association Study);
calculating the second relevance parameters β_{2(Fn),i};
calculating the second value PRS2(Fₙ) of polygenic risk score as a weighted sum of the alleles ALᵢ corresponding to the Single Nucleotide Polymorphisms SNPs of the second personalized subset SI₂, each weighted by the respective second relevance parameter β_{2(Fn),i}.

According to an implementation option, the method comprises the further steps of providing the processing results by means of a second table T₂ containing, for each of the Single Nucleotide Polymorphisms SNPsᵢ, the respective identifier ID_SNPᵢ, the respective effective allele or the respective two effective alleles (AL′_{1,i}, AL′_{2,i}) and the respective second calculated weight parameter (β_{1(Fn),i}); then, evaluating the predictive efficacy of each of the second values PRS2(Fₙ) of calculated polygenic risk score.

According to an implementation example, the LD-Pred algorithm modifies the weight of the association of the single SNP with the phenotype considering the degree of association between the SNPs themselves (Linkage Disequilibrium).

According to another implementation example, the SCT algorithm performs a penalized logistic regression regularized through the “elastic net” method (a combination of Lasso and Ridge regularizations, known per se) and which optimizes the parameters β to maximize the predictive capabilities of the model in terms of the phenotype.

In accordance with an embodiment of the method, the step of calculating the menopause onset probability, in relation to each of the at least one binary phenotype F_{Dj} and/or at least one continuous phenotype F_{Ck} considered, comprises calculating the menopause onset probability in relation to the at least one continuous phenotype P(F_{Ck}) based on the aforesaid first value PRS1(F_{Ck}) of polygenic risk score; and calculating the menopause onset probability in relation to the at least one binary phenotype P(F_{Dj}) based on a selection from the first value PRS1(F_{Dj}) of polygenic risk score, calculated by the LD-Fred algorithm, and the second value PRS2(F_{Dj}) of polygenic risk score.

According to an implementation option, the selection between the first value PRS1(F_{Dj}) of polygenic risk score and the second value PRS2(F_{Dj}) of polygenic risk score, for each of the binary phenotypes F_{Dj}, is carried out based on the evaluation of the respective predictive efficacy of each of the first values PRS1(F_{Dj}, ρ_{w}) of polygenic risk score and the second value PRS2(F_{Dj}) of polygenic risk score.

To optimally combine the results of the two algorithms and evaluate the “predictive effectiveness (of efficacy)” thereof, so as to choose the best result, or, in other words, to optimize the “measure of the ability to discern cases”, some methodologies can be used, known per se, based on the calculation of “meta-PRS”, which combine several PRS.

In this regard, the well-known “Inverse Variance Weighting” method (https://en.wikipedia.org/wiki/inverse-variance weighting) can be used, which provides weighing each variable, in this case each of the two PRS, in inverse proportion to the variance thereof.

According to an embodiment of the method, the step of calculating the menopause onset probability based on the respective first value PRS1(Fₙ) and/or second value PRS2(Fₙ) of polygenic risk score determined, comprises calculating the menopause onset probability, for each phenotype Fₙ, based on a respective relationship and/or empirical curve A3 which describes a statistical link between polygenic risk score values PRS and menopause onset probability.

According to an implementation option, each of such relationships and/or empirical curves A3 is obtained during a training step of the algorithms.

According to an implementation option, each of such relationships and/or empirical curves A3 is obtained based on a processing or interpolation of known data about the relationship between menopause onset and PRS of women whose menopause onset age and PRS are known.

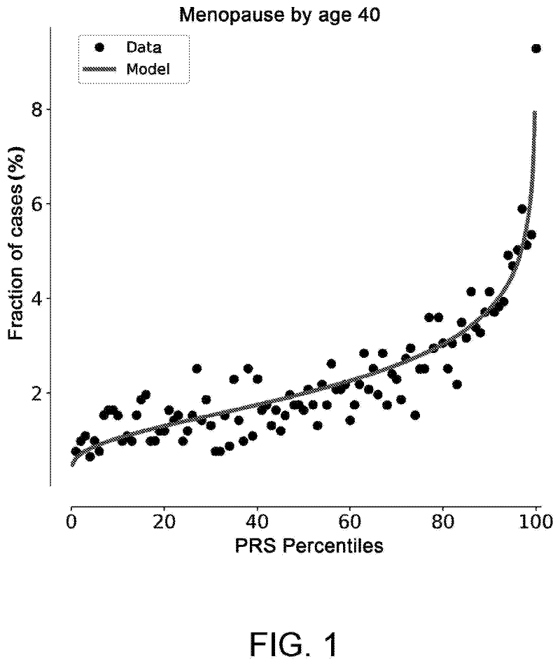

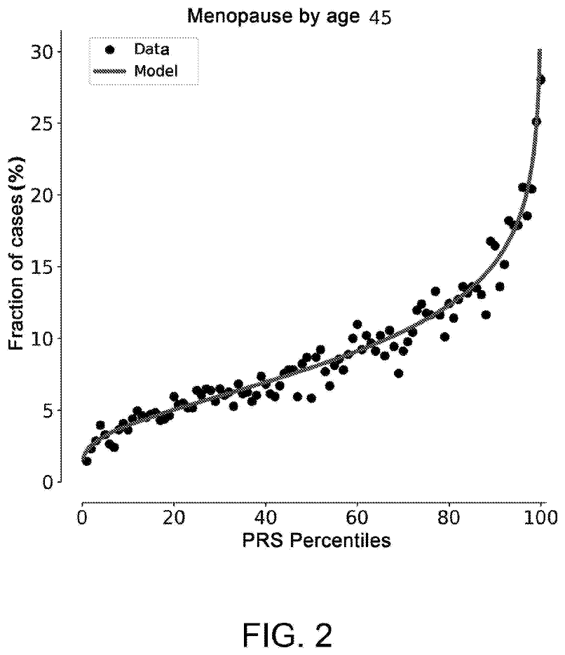

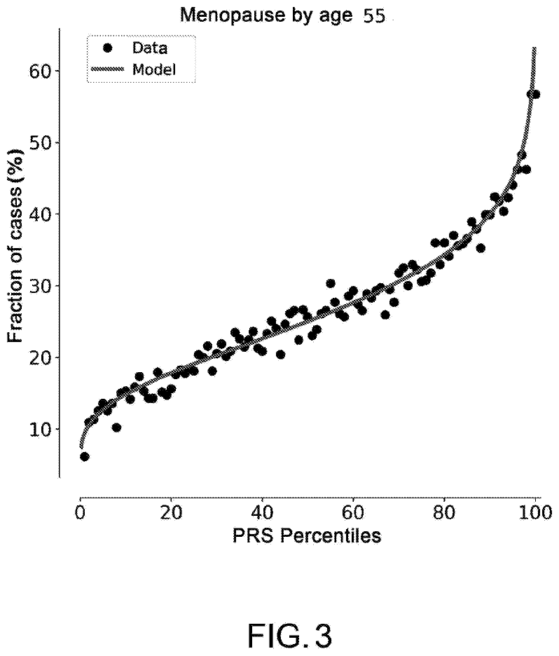

FIGS. 1-3 show “case fraction diagrams” illustrating the stratification of the population by percentage of cases found in each percentile of the Polygenic Risk Score, for a corresponding respective phenotype. The Polygenic Risk Score PRS is calculated for each of the individuals belonging to the “test dataset”. Based on this distribution, individuals are grouped into subsets (bins) containing individuals who are in the same PRS percentile. The percentage of individuals affected by the corresponding phenotype was calculated within the bin population.

The PRS percentile is indicated in the abscissa of the graphs. The fraction of the cases is shown in the ordinate of the graphs (i.e., the ratio of the number of people on which the phenotype was actually found to the number of people in the bin).

In each graph, the experimental percentages of individuals for which menopause onsets are indicated as points, for each bin associated with a PRS percentile, and a curve is also shown which approximates the trend of the experimental points and forms the aforesaid empirical curve A3.

In these graphs it can be observed, among other things, how the fraction of cases increases as the PRS increases.

As previously noted, according to an embodiment, the method comprises, before using the algorithms of the aforesaid set of algorithms, the further step of training the set of trained predictive algorithms, based on two subsets of a dataset containing genetic data of Single Nucleotide Polymorphisms SNPs of women whose menopause onset age is known: a first subset is used as a training dataset and the second subset is used as a validation database.

Some further details on the training and/or validating steps of an embodiment of the method are provided below, merely by way of non-limiting example.

The previously mentioned “UK BioBank” was used as a database.

Filters were developed on the data of such “UK BioBank” database to identify the women who began menopause due to natural causes and the menopause age was identified for these women. Based on these data, four different phenotypes were constructed, namely the aforementioned “Menopause before age 40”, “Menopause before age 45”, “Menopause after age 55”, and “continuous phenotype indicating the precise age of menopause”.

For each of these phenotypes, PRS were developed with the following methodology.

The degree of association (Linkage disequilibrium) between the genetic variants was calculated and for this purpose the publicly available “whole genome sequencing” data in the “1000 Genomes” project was used.

The “Linkage disequilibrium” represents the degree of association between the different SNPs, i.e., the probability of finding a variant in the presence of another.

For example: assume that the C allele is present in the position with id: rs17778; it has been found that, when this occurs, in 98% of cases the allele A is present in the allele rs17776. Thus, the two SNP are correlated with each other, and this is precisely the “Linkage disequilibrium”.

The reference dataset was divided into two subsets, one used as a “training dataset” (input for the LD-Pred and SCT algorithms) and one used as a “test dataset” for the subsequent validation procedures.

The output generated by the LD-Pred algorithm consists of a series of matrices containing a subset of SNP, among those present in the “summary statistics”, i.e., the SNP selected according to the following criteria (or, which have passed the following quality controls): Minor Allele Frequency >0.01 and non-ambiguity; furthermore, SNP including A/T or C/G as variants are excluded because they could present “strand” errors.

For the SNPs selected as indicated above, the weight parameters β are varied based on the fraction of causal variants p used and the Linkage Disequilibrium radius adopted.

The output generated by the SCT algorithm is a single PRS matrix.

The parameter “fraction of causal variants” p represents the fraction of causal SNP, i.e., how many SNPs out of the summary statistics total can be considered directly correlated with the phenotype and not correlated, by virtue of the presence of “Linkage Disequilibrium” with other SNPs. By changing this parameter, the PRS is changed and therefore the predictivity thereof.

As a measure of the ability to discern the “cases” (i.e., the people with the studied phenotype) from the “controls” (i.e., the people without it), the area under the “Receiving Operating Characteristic” (ROC-AUC) curve was used. This value is an index ranging from 0 to 1.

In practice, values between 0.5 and 1 are used (with 0.5 being the worst possible case in the prediction, equivalent to using a causal variable as a predictor). Any ROC-AUC values between 0 and 0.5 are interpreted as a possible error in the choice of data.

The ROC-AUC is used to verify the PRS results on the validation dataset.

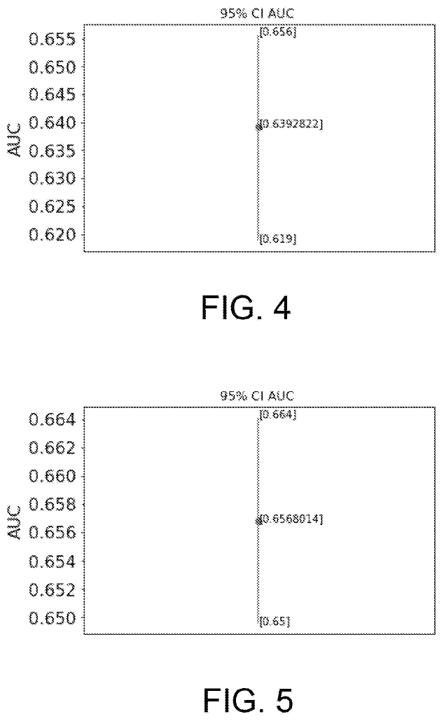

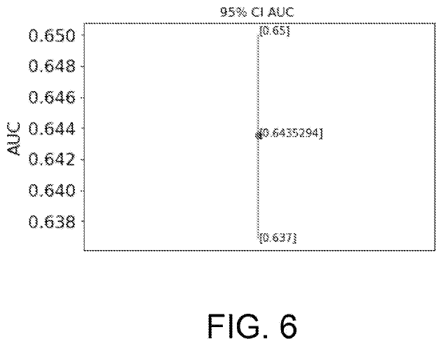

Examples of AUC resulting from the application of the ROC-AUC methodology are shown in FIGS. 4-6.

In such figures, the ROC-AUC graphs were calculated by testing the parameters β obtained as output in a logistic regression model with the related event (e.g., menopause before age 45) as a response variable and the PRS related to each individual of the “test dataset” as a predictor variable. The model was repeated 100 times for each type and the graphs in FIGS. 4-6 show the average value of the AUC and the confidence interval, respectively, for the phenotypes “menopause before age 40”, “menopause before age 45”, “Menopause after age 55”.

The LD-Pred algorithm was experimented with different values of ρ (1, 0.3, 0.1, 0.03, 0.01, 0.003, 0.0001) and the result with a greater ROC-AUC was that related to the phenotype “Menopause before age 45” with a ρ=0.01 (0.64).

The SCT algorithm was experimented with a filter which eliminates all the SNPs which have, in the GWAS summary statistics, a “P-value” parameter greater than 0.6, obtaining an AUC of 0.66, again for the “Menopause before age 45” phenotype.

A series of SNP matrices and weight β parameters were then developed to help calculating the PRS to identify the risk of early menopause.

Although the tables containing the relevant SNP sets and the associated weight parameters have a relative value and scope (since they depend both on the phenotype considered, among the many possible ones, and on the ancestral nature of the population, as previously explained), some extracts from such tables are nevertheless reported hereinafter, merely by way of example. It should be noted that such extracts, although very limited, since they show a few rows among the very many (around hundreds of thousands) of the complete tables obtained, are shown only to provide an example of how the tables appear, in which each row comprises a set of three associated information items “SNP identifier—effective allele—calculated significance parameter β”.

| | Effective | |
|---|---|---|
| SNP | Allele | BETA |
| rs16991615 | A | −0.25816857552715194 |
| rs11668344 | G | 0.21518220113721104 |
| rs7275360 | C | −0.181293048590242 |
| rs2720044 | C | −0.143316211460856 |
| rs4246511 | C | 0.10685772648691699 |
| rs365132 | T | −0.0905183436671588 |
| rs1713460 | G | 0.088173308856248 |
| rs4693089 | G | −0.0757630272643996 |
| rs349306 | A | −0.0728216782185266 |
| rs1799949 | A | −0.0701142714842333 |
| rs17474422 | C | −0.0691252126921703 |
| rs763121 | G | 0.06886853901606099 |
| rs10957156 | A | 0.0682766775309774 |
| rs16888892 | A | −0.066546381061405 |
| rs2835343 | G | 0.0649726800176715 |
| rs16888373 | A | 0.0645505657454957 |
| rs1507865 | C | 0.0628493598913269 |
| rs6572296 | T | −0.0626191298151644 |
| rs539412 | T | −0.0618566503624738 |
| rs6856693 | G | −0.0611760760321194 |
| . . . | . . . | . . . |

Excerpt from an example of a table obtained with the present method for the “menopause before age 40” phenotype, based on the first PRS score calculated with SCT.

| | Effective | |
|---|---|---|
| SNP | Allele | BETA |
| rs16991615 | A | 0.48937478621904706 |
| rs2277339 | G | −0.155897514226893 |
| rs11668344 | G | −0.11866251924589301 |
| rs365132 | T | 0.106856433783431 |
| rs4693089 | G | 0.0930515836758072 |
| rs6899676 | G | 0.0806595907199027 |
| rs1046089 | A | −0.0771526519929504 |
| rs6856693 | G | 0.0744412669406458 |
| rs1800932 | G | 0.0736646406897279 |
| rs2720044 | C | 0.0716238696469267 |
| rs704795 | A | −0.0707806111594526 |
| rs16858210 | A | 0.0701506961626314 |
| rs349306 | A | 0.0662648101505547 |
| rs7642211 | G | −0.0645700411993638 |
| rs13040088 | G | −0.0614475271478685 |
| rs2236553 | T | 0.0614475271478685 |
| rs16888892 | A | 0.0587653748993031 |
| rs8070740 | G | 0.0581496652898814 |
| rs1507865 | C | −0.0555006318493418 |
| rs236189 | G | −0.0547168448166173 |
| . . . | . . . | . . . |

Excerpt from an example of a table obtained with the present method for the “menopause after age 55” phenotype, based on the first PRS score calculated with SCT

| | Effective | |
|---|---|---|
| SNP | Allele | BETA |
| rs16991615 | A | −0.369082929361303 |
| rs2720044 | C | −0.15787557615909198 |
| rs11668344 | G | 0.13123432785597502 |
| rs4693089 | G | −0.11554243461084401 |
| rs4246511 | C | 0.10913116701638302 |
| rs6899676 | G | −0.0992545291124773 |
| rs1046089 | A | 0.0949391148032391 |
| rs6856693 | G | −0.0920491278061069 |
| rs17474422 | C | −0.0804147917804023 |
| rs763121 | G | 0.0802911750645638 |
| rs365132 | T | −0.0733868892974348 |
| rs4956542 | T | −0.06835257301334201 |
| rs11031006 | A | −0.067972666395388 |
| rs13040088 | G | 0.0674975102200367 |
| rs2236553 | T | −0.0674975102200367 |
| rs2277339 | G | 0.066982862160349 |
| rs930036 | A | 0.0665322651703257 |
| rs13008890 | A | 0.0654387499570415 |
| rs2236918 | G | −0.0640185778044724 |
| rs17007138 | C | 0.0633321991941702 |
| . . . | . . . | . . . |

Excerpt from an example of a table obtained with the present method for the “menopause before age 45” phenotype, based on the first PRS score calculated with SCT

| | Effective | |
|---|---|---|
| SNP | Allele | BETA |
| rs299163 | A | 0.15100999999999998 |
| rs11668344 | A | −0.07942 |
| rs2384687 | A | −0.073013 |
| rs349306 | A | −0.062479 |
| rs4806656 | A | −0.055255 |
| rs2547362 | T | −0.054821 |
| rs897798 | A | −0.05426 |
| rs4806660 | T | −0.05193 |
| rs236116 | A | 0.050098000000000004 |
| rs6053812 | A | 0.048277 |
| rs1172818 | T | 0.048 |
| rs12984783 | A | 0.03954 |
| rs17640544 | T | −0.036447 |
| rs2236553 | T | −0.035914999999999996 |
| rs299189 | A | −0.035671 |
| rs2277339 | T | −0.03335 |
| rs11084392 | T | 0.03157 |
| rs8101859 | A | 0.031508 |
| rs546082 | T | 0.029708 |
| rs3865476 | A | 0.028607 |
| . . . | . . . | . . . |

Excerpt from an example of a table obtained with the present method for the continuous phenotype “onset age” based on the first PRS score calculated with LD-Pred.

In addition to using the PRS of each phenotype as a single risk factor, all the PRS/Phenotype combinations were tested. For example, to predict the prognosis “Menopause before age 45”, the model obtained by combining the PRS related to the phenotype “Menopause before age 45” in combination with that related to “Menopause after age 55” was used.

Another important aspect and advantage of the method shown here is the possibility of using it in combination with already known models.

In such a case, the PRS is a further risk factor, calculated by the present method. Hence, known models can be used to estimate the risk of, for example, early menopause (cause of infertility) and add the PRS thereto to make them capable of providing more accurate predictions.

In particular, according to an embodiment, the method comprises carrying out a predictive prognosis of a woman's menopause onset based on the first value PRS1 (Fₙ) of polygenic risk score PRS and/or the second value PRS2(Fₙ) of polygenic risk score (PRS) in combination with further known risk factors.

According to an implementation option, the method comprises the steps of adding the first value PRS1(Fₙ) of polygenic risk score (PRS) and/or the second value PRS2(Fₙ) of polygenic risk score PRS, calculated as an additional risk factor within any known procedure of predictive prognosis of menopause based on risk factors other than said first value PRS1(Fₙ) of polygenic risk score PRS and/or second value PRS2(Fₙ) of polygenic risk score PRS, so as to obtain an improved procedure of predictive prognosis of menopause.

Lastly, the step of obtaining a predictive prognosis of menopause by means of said improved procedure of predictive prognosis of menopause is provided.

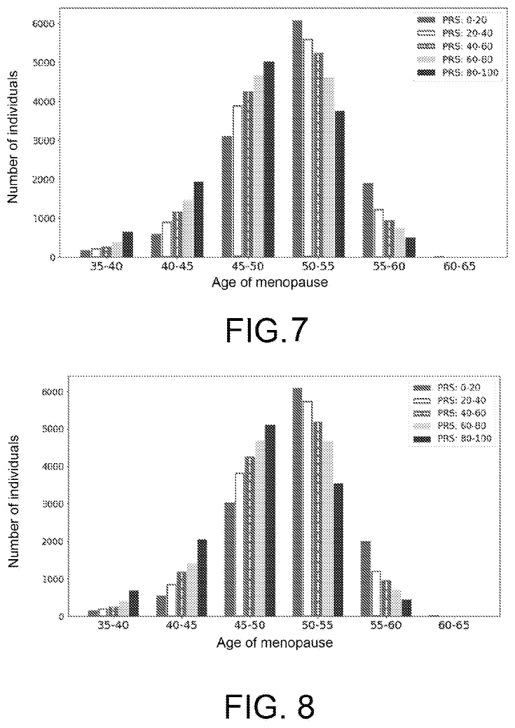

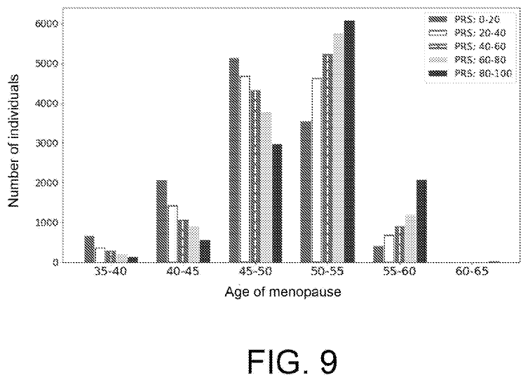

Regarding the form in which the results of the predictive prognosis can be presented, let us consider the further examples shown in FIGS. 7-9.

FIGS. 7-9 show “population distribution” diagrams, respectively for the phenotypes “menopause before age 40”, “menopause before age 45”, “menopause after age 55”. Such population distribution graphs were obtained by grouping the “test dataset” population within 5-year groups by menopause age. Each column shows a grouping of 20 PRS percentiles.

The aforesaid population distribution bar charts were obtained by grouping the “test dataset” population within 5-year groups by menopause age.

The ordinate shows the number of individuals found in the reference dataset.

In each of the diagrams (therefore for each of the phenotypes associated with the respective diagram) the age groups are indicated on the abscissa. Five columns are shown for each age group, representing a grouping of 20 PRS percentiles (quintile), namely, from left to right: PRS 0-20; PRS 20-40; PRS 40-60; PRS 60-80; PRS 80-100. The height of each column represents the number of women who began menopause at the corresponding age indicated in the abscissa having the PRS in the respective quintile. Inter alia, these graphs show that, for the phenotypes before age 40 and before age 45, women with a PRS in the last quintile (80-100) represent the largest fraction in the first three age groups, associated with menopause onset at a younger age, while in the last two groups, associated with menopause onset at a later age, there are more women with PRS in the first quintile (0-20).

A similar phenomenon, but of the opposite sign, can be observed in the menopause phenotype over age 55: in this case, women with a high PRS score (fifth column 80-100) are mainly represented in the last three groups (menopause after age 50).

These graphs therefore support the evidence of the ability of the PRS score to stratify the population of women according to the age at which menopause is reached.

According to an embodiment of the method, the results can be read inversely, i.e., interpreting the absence of an effective allele as a positive factor against the risk of menopause onset. It is therefore possible to carry out weighted sums, for the purpose of calculating the PRS, with positive or negative relevance parameters.

From the perspective of practical implementation, a medical device can be used to calculate the PRS for each patient, for example a medical device registered by Allelica, after updating it to add the menopause PRS.

Using this methodology, after genotyping the person in question through the use of a MicroArray or “whole genome sequencing”, it is possible to directly calculate the PRS and thus identify the relative risk of early menopause.

As already noted, it is further possible to update any model adapted to predict the absolute risk of beginning early menopause by adding the PRS as a covariate and thus obtaining an improvement in the predictivity thereof.

As can be seen, the objects of the present invention, as previously indicated, are fully achieved by the method described above, by virtue of the features disclosed above in detail.

In fact, the method allows to have “optimized” subsets of SNPs and relevance parameters that are recalculated (compared to those known, for example, of GWAS) by means of appropriate training of appropriate algorithms, as indicated above, such as to provide predictions which have proven effective in terms of reliability, accuracy, and precision, based on numerous validation tests carried out.

Furthermore, the results can be provided with respect to a plurality of phenotypes of interest (including those previously mentioned).

The PRS calculated by the method according to the present invention provides the ability to discern cases from controls, allowing to describe the relative (genetic) risk of beginning menopause in a certain age group, for example, before age 45.

By carrying out this test on a woman before age 35, it is therefore possible to identify the risk of a decline in fertility already underway in order to be able to plan her reproductive life, even by resorting to programmed fertility interventions such as egg vitrification.

Furthermore, the PRS is a risk factor transversal to other risk factors, it is therefore possible to use it together with the other indices to obtain an even more accurate result of the risk of early menopause.

In order to meet contingent needs, those skilled in the art may make modifications and adaptations to the embodiments of the method described above and can replace elements with others which are functionally equivalent without departing from the scope of the following claims. All the features described above as belonging to one possible embodiment may be implemented irrespective of the other embodiments described.

1-22. (canceled)

23. A computer-implemented method for a predictive prognosis of menopause onset in a woman, comprising the steps of:
accessing a woman's genetic data comprising Single Nucleotide Polymorphisms (SNPs) of the woman;
processing said woman's genetic data to obtain a predictive prognosis of menopause onset in relation to at least one phenotype comprising at least one age group or an age limit with respect to which the predictive prognosis of menopause onset is to be carried out, or an indication of a woman's age at which the menopause onset is more likely;
providing as a result said predictive prognosis of menopause onset in relation to said at least one phenotype;
wherein for each phenotype of said at least one phenotype, the processing step comprises the steps of:
identifying, in the woman's genetic data, a first personalized subset of Single Nucleotide Polymorphisms which also belong to a first predetermined set, associated with the phenotype considered, wherein each of the Single Nucleotide Polymorphisms of said first predetermined set comprises an identifier of Single Nucleotide Polymorphisms, and is associated with a respective pre-calculated first relevance parameter;
calculating a first value of polygenic risk score, based on said first custom subset of Single Nucleotide Polymorphisms and the respective pre-calculated first relevance parameters;
determining the predictive prognosis of menopause onset in relation to each of said at least one phenotype, based on the respective first value of polygenic risk score;
wherein the determination of said first predetermined set of Single Nucleotide Polymorphisms and the calculation of said first relevance parameters are carried out in a preliminary training step, which is independent from said step of accessing woman's genetic data and prior to said step of processing the woman's genetic data,
said preliminary training step comprising training at least one algorithm using machine learning and/or artificial intelligence techniques, on the basis of known data, said training being carried out based on a known dataset containing genetic data of Single Nucleotide Polymorphisms of women whose menopause onset age is known.

24. The method according to claim 23, wherein:
said identifier of Single Nucleotide Polymorphisms comprises a genetic variant address and an effective allele present in said genetic variant address;
said first custom subset comprises Single Nucleotide Polymorphisms in which said effective allele is traced as present in the respective genetic variant address, in the woman's genetic data, and is associated with a respective allele dosage;
said step of calculating a first value of polygenic risk score comprises summing up the value of all the pre-calculated first relevance parameters associated with all the respective Single Nucleotide Polymorphisms of the first personalized subset, each multiplied by the respective allele dosage of the effective allele.

25. The method according to claim 23, wherein the preliminary training step comprises building the first predetermined set of Single Nucleotide Polymorphisms by of a selection of relevant Single Nucleotide Polymorphisms carried out through the following steps:
identifying Single Nucleotide Polymorphisms statistically associated with the phenotype through a genetic association study, each of said Single Nucleotide Polymorphisms identified being associated with a respective known initial relevance parameter;
identifying optimal values of first relevance parameters adapted to optimize the predictive efficacy of the first value of polygenic risk score, wherein said optimization of the predictive efficacy of the first value of polygenic risk score is carried out from said identified Single Nucleotide Polymorphisms and respective known initial relevance parameters, through the single or combined use of one or more predictive algorithms,
wherein each of said predictive algorithms is trained based on a known dataset containing genetic data of Single Nucleotide Polymorphisms of women whose menopause onset age is known;
defining said first predetermined set of Single Nucleotide Polymorphisms based on said identified Single Nucleotide Polymorphisms;
defining as respective first relevance parameters said respective identified optimal values of the first relevance parameters.

26. The method according to claim 25, wherein said one or more of the following predictive algorithms comprise one or more of the following algorithms:
Clumping+Thresholding;
LD-Pred;
Stacked Clumping+Thresholding.

27. The method according to claim 23, wherein the results of the predictive prognosis comprise a menopause onset probability, in relation to at least one phenotype,
wherein said step of determining a predictive prognosis comprises calculating a menopause onset probability with respect to each of the phenotypes considered,
and wherein said step of providing as a result the predictive prognosis of menopause onset comprises providing as a result of the prognosis the calculated menopause onset probability, in relation to the at least one phenotype.

28. The method according to claim 23, wherein said at least one phenotype comprises a plurality of phenotypes comprising at least one binary phenotype and at least one continuous phenotype,
wherein the at least one binary phenotype comprises at least one age group or an age limit with respect to which the prognosis of menopause onset or not is to be determined, and the at least one continuous phenotype comprises an indication of a woman's age at which the menopause onset will be more likely;
the step of determining the predictive prognosis of menopause onset comprises determining the predictive prognosis of menopause onset in relation to each of said at least one binary phenotype and at least one continuous phenotype, based on the respective first value of polygenic risk score;
the step of providing as a result the predictive prognosis of menopause onset comprises providing the predictive prognosis of menopause onset both in relation to each of the at least one binary phenotype and in relation to each of the at least one continuous phenotype.

29. The method according to claim 23, wherein the at least one binary phenotype comprises one or more phenotypes belonging to the following group:
age of menopause onset before age 40, corresponding to Primary Ovarian Insufficiency;
age of menopause onset before age 45, corresponding to Early Menopause;
age of menopause onset after age 55, corresponding to Late Menopause;
and wherein the at least one continuous phenotype comprises an indication of the age at which the menopause onset is estimated as more likely and/or a distribution of menopause onset probability in relation to each year of the woman's age within a predetermined age range.

30. The method according to claim 23, comprising, after the step of identifying a first personalized subset of Single Nucleotide Polymorphisms, a step of selecting a first group of Single Nucleotide Polymorphisms, belonging to said first personalized subset of the woman, comprising a first number of Single Nucleotide Polymorphisms recognized as the most relevant ones based on a predetermined criterion, and
wherein the step of calculating a first value of polygenic risk score comprises calculating the first value of polygenic risk score based on said first group of Single Nucleotide Polymorphisms and respective pre-calculated first relevance parameters.

31. The method according to claim 30, wherein the relevant Single Nucleotide Polymorphisms are selected according to one of the following criteria:
identifying as the most relevant Single Nucleotide Polymorphisms those Single Nucleotide Polymorphisms which are associated with the highest relevance parameter values; or
testing different polygenic risk scores calculated on different Single Nucleotide Polymorphisms, validating the polygenic risk scores on known populations, and choosing those Single Nucleotide Polymorphisms which result in a better predictivity using as a metric sensitivity comprising an ability to identify people affected by the disease as people at risk, or specificity comprising an ability to identify unaffected people as people that are not at risk; or
applying AUC-ROC (Area Under the Receiver Operator Characteristic Curve) methodologies.

32. The method according to claim 23, comprising the further steps of:
identifying, in the woman's genetic data, a second personalized subset of Single Nucleotide Polymorphisms which also belong to a second predetermined set, associated with the phenotype considered, wherein each of the Single Nucleotide Polymorphisms of said second predetermined set comprises an identifier of Single Nucleotide Polymorphisms, and is associated with a respective pre-calculated second relevance parameter; wherein said identifier of Single Nucleotide Polymorphisms comprises a genetic variant address and an effective allele present in such a genetic variant address;
calculating a second value of polygenic risk score, based on said second personalized subset of Single Nucleotide Polymorphisms and the respective pre-calculated second relevance parameters;
wherein the determination of said second predetermined set of Single Nucleotide Polymorphisms and the calculation of said second relevance parameters are carried out in a preliminary training step, which is independent from said step of accessing the woman's genetic data and prior to said step of processing the woman's genetic data, said preliminary training step comprising training at least one algorithm by machine learning and/or artificial intelligence techniques, based on known data;
wherein the step of calculating the menopause onset probability comprises calculating the menopause onset probability in relation to each of said at least one phenotype, based on the respective first value of polygenic risk score and/or the respective second value of polygenic risk score,
and wherein the menopause onset probability in relation to at least one phenotype of said at least one phenotype is calculated based on both the respective first value of polygenic risk score and the respective second value of polygenic risk score.

33. The method according to claim 23, wherein the step of calculating the menopause onset probability based on the respective determined first value and/or second value of polygenic risk score comprises calculating the menopause onset probability, for each phenotype, based on a respective relationship and/or empirical curve which describes a statistical link between polygenic risk score values and menopause onset probability.

34. The method according to claim 32, wherein the menopause onset probability in relation to at least one phenotype is calculated based on a weighted combination of the respective first value of polygenic risk score and second value of polygenic risk score.

35. The method according to claim 32, comprising, after the step of identifying a second personalized subset of Single Nucleotide Polymorphisms, a step of selecting a second group of Single Nucleotide Polymorphisms, belonging to said second personalized subset of the woman, comprising a second number of Single Nucleotide Polymorphisms recognized as the most relevant ones based on a predetermined criterion,
and wherein the step of calculating a second value of polygenic risk score comprises calculating the second value of polygenic risk score based on said second group of Single Nucleotide Polymorphisms and the respective second pre-calculated relevance parameters.

36. The method according to claim 32, wherein:
said first number of relevant SNPs is at least 10;
said second number of relevant SNPs is at least 10.

37. The method according to claim 23, wherein the polygenic risk scores calculated for one of the discrete phenotypes are used as additional information to establish the menopause onset probability referred to another phenotype together with the polygenic risk scores calculated for said phenotype, according to any combination of phenotypes and polygenic risk scores.

38. The method according to claim 37, wherein said at least one predictive algorithm comprises a first trained LD-Pred algorithm, configured to perform, based on a preventive training procedure, and for each of the phenotypes, the following steps:
determining said first predetermined set of Single Nucleotide Polymorphisms, containing said first plurality of Single Nucleotide Polymorphisms identified as relevant with respect to the menopause onset, based on a known set of Single Nucleotide Polymorphisms identified by GWAS (Genome Wide Association Study);
calculating said first relevance parameters taking into account a degree of association (Linkage Disequilibrium) between the Single Nucleotide Polymorphisms, and a parameter representative of the fraction of Single Nucleotide Polymorphisms identified as random for the phenotype considered; wherein, for each of the first parameters, a plurality of values is calculated, each of which corresponds to a respective parameter value representative of the fraction of, representative of a fraction of random variants used, belonging to a plurality of predetermined values of said parameter representative of a fraction of random variants used;
calculating the first value of polygenic risk score for each of said values of the parameter representative of the fraction of SNP, as a weighted sum of the alleles corresponding to the Single Nucleotide Polymorphisms of said first personalized subset, each weighted by the respective first parameter;
or wherein said at least one predictive algorithm comprises at least one second trained SCT algorithm, configured to perform, based on a preventive training procedure, and for each of the phenotypes, the following steps:
determining said second predetermined set of Single Nucleotide Polymorphisms, containing said first plurality of Single Nucleotide Polymorphisms identified as relevant with respect to the menopause onset, based on a known set of Single Nucleotide Polymorphisms identified by GWAS (Genome Wide Association Study);
calculating said second relevance parameters;
calculating the second value of polygenic risk score as a weighted sum of the alleles corresponding to the Single Nucleotide Polymorphisms of the second custom subset, each weighted by the respective second relevance parameter.

39. The method according to claim 25, wherein the step of calculating the menopause onset probability in relation to each of said at least one binary phenotype and at least one continuous phenotype comprises:
calculating the menopause onset probability in relation to the at least one continuous phenotype based on said first value of polygenic risk score;
calculating the menopause onset probability in relation to the at least one binary phenotype based on a selection between the first value of polygenic risk score and the second value of polygenic risk score.

40. The method according to claim 39, wherein the selection between the first value of polygenic risk score and the second value of polygenic risk score, for each of the binary phenotypes, is carried out based on the evaluation of the respective predictive efficacy of each of the first values of polygenic risk score and the second value of polygenic risk score.

41. The method according to claim 23, comprising, before using the algorithms of the aforesaid set of algorithms, the further step of training the set of trained predictive algorithms, based on two subsets of a dataset containing genetic data of Single Nucleotide Polymorphisms of women whose menopause onset age is known, a first subset being used as a training dataset and a second subset being used as a validation dataset.

42. The method according to claim 23, comprising carrying out a predictive prognosis of a woman's menopause onset based on the first value of polygenic risk score and/or the second value of polygenic risk score in combination with further known risk factors,
or wherein the method further comprises the steps of:
adding the first value of polygenic risk score and/or the second value of polygenic risk score calculated as an additional risk factor within any known procedure of predictive prognosis of menopause based on risk factors other than said first value of polygenic risk score and/or second value of polygenic risk score, to obtain an improved procedure of predictive prognosis of menopause;
obtaining a predictive prognosis of menopause by said improved procedure of predictive prognosis of menopause.
